(19) United States
(12) Patent Application Publication
(10) Pub. No.: US 2008/0051777 A1
(43) Pub. Date: Feb. 28, 2008

(54) RADIOFREQUENCY ABLATION DEVICE FOR REDUCING THE INCIDENCE OF SKIN BURNS

(76) Inventor: Dieter Haemmerich, Charleston, SC (US)

Correspondence Address: PARKS KNOWLTON, LLC, 1117 Perimeter Center West, Suite E402, Atlanta, GA, 30338, US

(21) Appl. No.: 11/846,293

(22) Filed: Aug. 28, 2007

Related U.S. Application Data
(60) Provisional application No. 60/823,726, filed on Aug. 28, 2006.

(51) Int. Cl. A61B 18/18

(52) U.S. Cl. 606/33

(57) ABSTRACT

A radiofrequency ablation device reduces the incidence of skin burns by selectively activating ground pads in a partially sequential and partially contemporaneous manner. The selective sequential activation of the ground pads lowers power dispersion at each ground pad relative to contemporaneous activation of ground pads and/or sequential activation of ground pads. This reduction in power dispersion at each ground pad consequently yields a lower overall maximum temperature, and more uniform heating of all of the ground pads. This makes the system more predictable and safer for use in treatment. The radiofrequency ablation device may include a layered ground pad that includes at least two layers of material. One layer of material can be substantially similar to the material used for a monolithic ground pad, and one layer can have a substantially lower relative electrical conductivity, but a substantially higher electrical conductivity relative to adjacent body tissue. Alternatively, a ground pad may include multiple conductive zones that are activated using the same selective sequential activation scheme.

### CROSS REFERENCE TO RELATED APPLICATIONS

This application claims priority to U.S. Provisional Application No. 60/823,726, filed Aug. 28, 2006, the entirety of which is herein incorporated by reference.

### TECHNICAL FIELD

The present invention relates generally to the field of electrosurgical devices. More particularly, the present invention relates to an advanced radiofrequency ablation device for reducing skin burns.

### BACKGROUND

Radiofrequency (“RF”) ablation is increasingly utilized as a minimally invasive treatment for primary and metastatic hepatic tumors, as well as tumors in kidney lung, bone, adrenal glands, and other areas of the body. Radiofrequency ablation is often used when surgery would entail high risk, or when surgery would be difficult or impossible.

During radiofrequency ablation, radiofrequency current is delivered to tissue via an electrode or electrodes. Electrodes are typically inserted directly into a tumor or other defined treatment area. The electrodes can be inserted percutaneously, laparoscopically, or during open surgery. Once the electrode is placed at the treatment site, a radiofrequency current can be applied to the electrode. The applied radiofrequency current is converted from electromagnetic energy to heat by ionic agitation. Heating to temperatures above approximately 50° C. can cause denaturation of intracellular proteins, destruction of tumor cell membranes, and eventually tumor cells necrosis.

In order to effect delivery of the radiofrequency current to tissue, there must be a return path for the applied current. Typically, a plurality of ground pads (dispersive electrodes) are applied to the skin of the patient receiving treatment to serve as a return path. A ground pad is often a flexible, thin-layered electrical conductor coated with an adhesive polymer that attaches to a patient's skin.

Since the introduction of radiofrequency ablation for tumor treatment, the size of the coagulation zone has been one major limitation in treatment of large tumors. Work has been done to increase the coagulation zone, most commonly by altering the design of the electrode, or by increasing radiofrequency generator power output.

Initially, radiofrequency ablation systems operated at around 25 Watts (W) of power and created coagulation zones with a diameter of approximately 1.5 cm. Current radiofrequency ablation systems operate at between 200-250 W of power and can create a coagulation zone with a diameter from about 4 cm to about 6 cm. A current goal is to continue increasing the coagulation zone, and to simultaneously decrease the required treatment time. Therefore, there is a continued interest in further increasing radiofrequency generator power output, since this can help achieve both of these goals simultaneously.

One problem with increasing radiofrequency generator power output is the problem of tissue heating below ground pads. As the maximum power output of radiofrequency generators has increased, the incidence of skin burns due to ground pad heating has also increased. Current statistics indicate that between about 0.1% and about 3.2% of patients receiving radiofrequency ablation treatment receive severe skin burns, defined as second- and third-degree burns. Between about 5% and about 33% of patients receiving radiofrequency ablation treatment report minor skin burns, defined as first-degree burns. However, some research suggests that the incidence of skin burns may be underreported.

The typical skin temperature of a human being at rest is around 33° C. First-degree skin burns can occur after approximately 10 minutes of temperatures measuring around 41° C. or higher. Meanwhile, second- and third-degree burns can occur in only a few seconds with temperatures greater than about 47° C., and about 52° C., respectively. Since skin burns begin to occur at fairly low temperatures, it is desirable to reduce the heating of the ground pads as much as possible.

In the art of radiofrequency ablation devices, it is known that current density is highest at the edges of ground pads. In particular, the “leading edge” of the ground pad, i.e., the edge of the ground pad proximal to the electrode, experiences the maximum current density and, consequently, the highest heating. There have been efforts to find ways to reduce heating of the ground pads, particularly the leading edge of the ground pads, in an attempt to reduce skin burns.

One method of reducing ground pad heating is to include a plurality of ground pads in an attempt to dissipate the heating over several surfaces. One commonly-used ground pad setup includes the use of four ground pads connected in parallel to a radiofrequency power source. Two of the four ground pads are attached to each thigh of the patient receiving treatment. When using two pads on each thigh, one pad can be attached to the anterior surface of a first thigh, and the second pad can be attached to the posterior surface of the first thigh. A similar configuration can be used on the second thigh. While including multiple ground pads is sound in theory, practical difficulties prevent such a technique from curing for the problem of ground pad heating.

First, the current tends to choose the path of least resistance at any given moment. Therefore, if the ground pads are not located a substantially equal distance from the electrode, the current tends to travel preferentially to the nearest electrode. Second, different tissues and materials are more or less conductive relative to each other. Since the electrode is typically placed somewhere about the patient's midsection, and the ground pads are often placed at or near the thighs, any intervening non-tissue material, such as a joint replacement or a prosthesis will further alter the travel path of the current. This makes placement of the electrodes even more difficult since merely measuring distances from the electrode to each of the ground pads will not necessarily be sufficient, even if the measurements are theoretically perfect. Finally, patients, electrodes, and/or ground pads can move during treatment. Therefore, even if the ground pads are theoretically perfectly uniformly spaced from the electrode before beginning the radiofrequency ablation treatment, it is possible that the ground pads' relative distances from the electrode can change during radiofrequency ablation treatment.

Another method used to reduce ground pad heating is cooling the ground pads. This method addresses the effects of a problem, namely preferential current dispersion at one ground pad, but this method does little or nothing to address the problem of preferential dispersion. Additionally, one commercially available radiofrequency ablation system provides monitoring of the current through each of the ground pads and alerts the user if there is uneven current distribution. As such, the user can adjust the placement of the ground pads to ensure that the ground pads are equidistant, with respect to each other, from the electrode. Another commercially available radiofrequency ablation device provides monitoring of skin temperatures at or near the leading edge of the ground pads. The device can alert the user if the skin temperature at the leading edge of a ground pad exceeds a safety threshold. These monitoring systems are able to shut-down the radiofrequency ablation device if skin temperature or current travel violates some predetermined threshold or rule. All of these methods can require operator interaction and can therefore raise the risk of operator error, and/or increase overall treatment times required for a radiofrequency ablation treatment session.

Another attempted method of reducing ground pad heating involves sequential activation of ground pads. This technique involves providing a set of ground pads, and providing power to each ground pad in turn at various intervals. In theory, sequential activation of ground pads would spread the current evenly among the ground pads, and would prevent the preferential delivery of current to the leading edge of the conductor proximal to the electrode. Therefore, in theory, sequential activation of grounding pads should equalize heating at each ground pad used. However, clinical research and trials revealed that sequential activation of ground pads actually results in an increase in ground pad heating relative to simultaneous activation of all of the ground pads. This increase in ground pad heating occurs due to a number of factors, including unintended arcing between ground pads, even if one of the pads is currently deactivated.

### SUMMARY OF THE INVENTION

A radiofrequency ablation device is provided that reduces the incidence of skin burns by selectively activating ground pads in a partially sequential and partially contemporaneous manner. While sequential activation of the ground pads was found to increase overall temperature of the leading edge of the ground pad proximal to the electrode, the selective sequential activation of the ground pads lowers power dispersion at each ground pad relative to cotemporaneous activation of ground pads and/or sequential activation of ground pads. This reduction in power dispersion at each ground pad consequently yields a lower overall maximum temperature, and more uniform heating of all of the ground pads. This makes the system more predictable and safer for use in treatment. Additionally, the more uniform heating and lower overall maximum temperature can enable the use of higher power in radiofrequency ablation. This can help increase the coagulation zone and decrease treatment times.

Another aspect of certain embodiments of the radiofrequency ablation device relates to the ground pads. In these embodiments, each ground pad is layered. A layered ground pad includes at least two layers of material, which may be disparate. For example, one layer of material can be substantially similar to the material used for a conventional ground pad, and another layer can have a substantially lower electrical conductivity relative to the conventional layer, but a substantially higher electrical conductivity relative to adjacent body tissue.

According to an embodiment of the present invention, a radiofrequency ablation device includes one or more electrodes that are located at an ablation site of a patient receiving radiofrequency ablation treatment. A multiple of conductors are located at a grounding site of a patient and are electrically coupled, through the patient, to the electrode. The electrode is coupled to a power supply. A switching circuit is coupled to the conductors. The switching circuit selectively activates or deactivates any number of the conductors in any desired combination. During a treatment session, the switching circuit will activate at least two of the conductors in combination for some period of time, and will deactivate at least one conductor for another period of time.

According to an aspect of the present invention, at least three conductors are included in the radiofrequency ablation device.

According to a further aspect of certain embodiments of the present invention, each conductor can include at least two layers of electrically conductive material, for example, a first layer and a second layer. The first layer of electrically conductive material can be more electrically conductive than the second layer of electrically conductive material, which can be located proximal to the skin of the patient receiving radiofrequency ablation treatment.

According to a further aspect of the present invention, the radiofrequency ablation device can also include a control system that is operatively coupled to the switching circuit. The control system executes computer readable instructions that control operation of the switching circuit.

According to a further aspect of the present invention, the radiofrequency ablation device can also include a monitoring system attached to or the ground pad.

According to a further aspect of the present invention, the radiofrequency ablation device can also include a control system operatively coupled to the switching circuit. The control system controls operation of the switching circuit based, at least partially, based on data obtained by the monitoring system.

In certain embodiments of the present invention, the monitoring system includes a temperature sensor.

In certain other embodiments, the monitoring system attached to the ground pad includes a current sensor.

In yet other embodiments, the monitoring system attached to the ground pad includes an impedance sensor.

In certain embodiments of the present invention, the switching circuit is a relay.

According to a further aspect of the present invention, the switching circuit activates or deactivates the conductors at zero-crossings of the radiofrequency signal.

The present invention also provides a method for reducing the incidence of skin burns during radiofrequency ablation treatment. The method includes locating at least one electrode at an ablation site of a patient receiving radiofrequency ablation treatment. Several conductors (ground pads) are operatively coupled to the electrode, and are located at respective grounding sites on the patient. A power supply can be electrically coupled to the electrode, and a switching circuit can be electrically coupled to the conductors. The switching circuit selectively activates or deactivates any number of the conductors in any desired combination. During a treatment session, the switching circuit will activate at least two conductors in combination for some period of time, and will deactivate at least one conductor for another period of time.

These and further features of the present invention will be apparent with reference to the following description and attached drawings.

### BRIEF DESCRIPTION OF THE DRAWINGS

FIG. 1 schematically illustrates a conventional radiofrequency ablation system attached to a patient for treatment.

FIG. 2 schematically illustrates a second view of a conventional radiofrequency ablation system attached to a patient for treatment.

FIG. 3 is a schematic illustration of exemplary activation times for three ground pads of a radiofrequency ablation device according to an exemplary embodiment of the present invention.

FIG. 4 is a partial sectional view of a monolithic ground pad for use with a radiofrequency ablation device according to an exemplary embodiment of the present invention.

FIG. 5 is a partial sectional of a layered ground pad for use with a radiofrequency ablation device according to an exemplary embodiment of the present invention.

FIG. 6 is a perspective view of an exemplary a multiple-zone ground pad according to an exemplary embodiment of the present invention.

### DETAILED DESCRIPTION

As required, detailed embodiments of the present invention are disclosed herein. It must be understood that the disclosed embodiments are merely exemplary examples of the invention that may be embodied in various and alternative forms, and combinations thereof. As used herein, the word “exemplary” is used expansively to refer to embodiments that serve as an illustration, specimen, model or pattern. The figures are not necessarily to scale and some features may be exaggerated or minimized to show details of particular components. In other instances, well-known components, systems, materials or methods have not been described in detail in order to avoid obscuring the present invention. Therefore, specific structural and functional details disclosed herein are not to be interpreted as limiting, but merely as a basis for the claims and as a representative basis for teaching one skilled in the art to variously employ the present invention.

As used in this specification, the terms “ground pad” and “conductor” are free interchangeable.

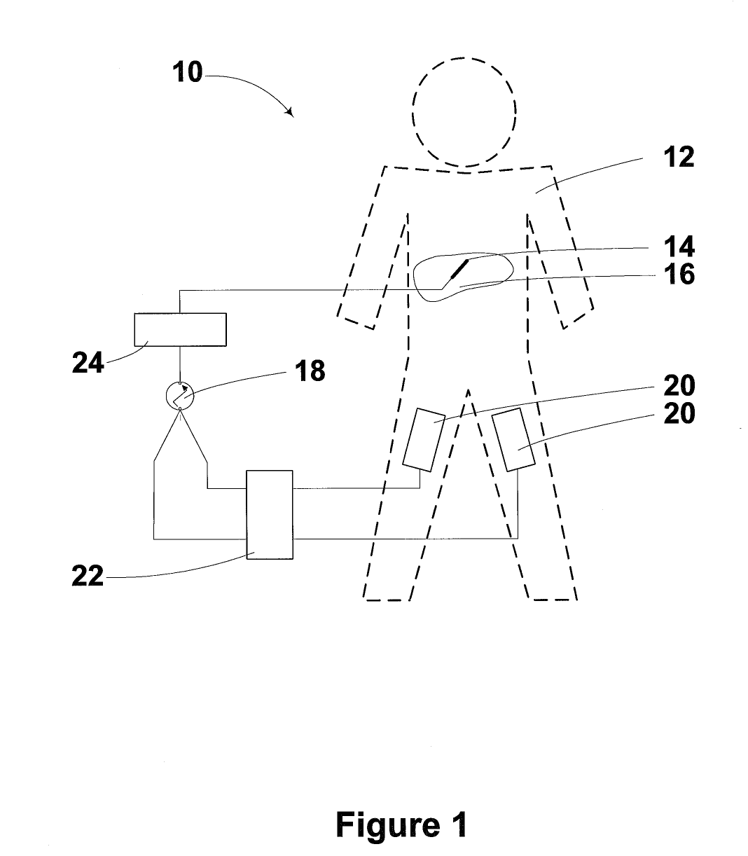

Referring initially to FIG. 1, a radiofrequency (RF) ablation device 10 is illustrated. In FIG. 1, the radiofrequency ablation device is attached to a patient 12 who is receiving treatment. An electrode 14 is inserted into a treatment region 16, typically a tumor site within the patient 12. In the illustrated example, the treatment region 16 is the patient's liver.

The electrode 14 is electrically coupled to a radiofrequency generator 18. The radiofrequency generator 18 is electrically coupled to at least one conductor 20. While the illustrated example includes two conductors 20, one on each thigh, it should be understood that any number of conductors 20 can be placed anywhere on, in, or in close proximity to the patient 12. Typically, the conductors 20 include an adhesive polymer and are placed in contact with the patient's skin. The adhesive polymer keeps the conductors 20 close to the patient's skin and assists in creating and maintaining an electrical coupling between the conductors 20 and the electrode 14.

Electrical power can be applied to the electrode 14 by generating radiofrequency power at the radiofrequency generator 18, and allowing the power to flow to the electrode 14 as an electrical current. The electrical current flows to the tumor site, where it causes resistive heating around the electrode 14. The resistive heating around the electrode 14 can cause the destruction of tumor cells. The current then disperses through the body of the patient 12 and travels to the conductors 20. The conductors 20 return the current to the radiofrequency generator 18, thereby completing the circuit.

In addition to the components described above, the radiofrequency system 10 can optionally include peripheral components 22, 24. The peripheral components 22, 24 can be located between the radiofrequency generator 18 and the conductors 20a, 20b, between the radiofrequency generator 18 and the electrode 14, or both. Each of the peripheral components 22, 24 can include one or more elements, such as, but not limited to, monitoring systems, coolers, heaters, alarms, control logic, computers, digital multimeters, data acquisition devices, auxiliary power sources, timers, imaging devices, combinations thereof, and the like.

It should be understood that the peripheral components 22, 24 can also include monitoring devices that extend to or into electrodes 14 and conductors 20. For example, a thermal monitoring system can be included as part or all of peripheral component 22. A thermal device, such as a thermocouple, can determine the temperature at a conductor 20 and report the temperature to the thermal monitoring system. Various functions can be set as trigger events based on a temperature threshold. For example, a temperature reading that exceeds a set threshold could trigger an automatic shut-off, an alarm, deactivation of the affected conductor, a combination thereof, or the like. In addition to operating as a trigger event, the temperature measured at each conductor 20 can be integrated into a feedback loop control system to control which conductors 20 are activated, as well as time of activation, in an effort to keep all conductor temperatures equal. Similarly, a current sensor or an impedance sensor can measure current to each conductor 20, or impedance between each conductor 20 and an electrode 14. Similarly to the exemplary use of temperature readings, a measurement of current or impedance that exceeds a set threshold can be used as a trigger event, or can be used in a feedback control system to determine activation of conductors 20. The control algorithm can be a PID algorithm, or any other control algorithms now known, or later developed.

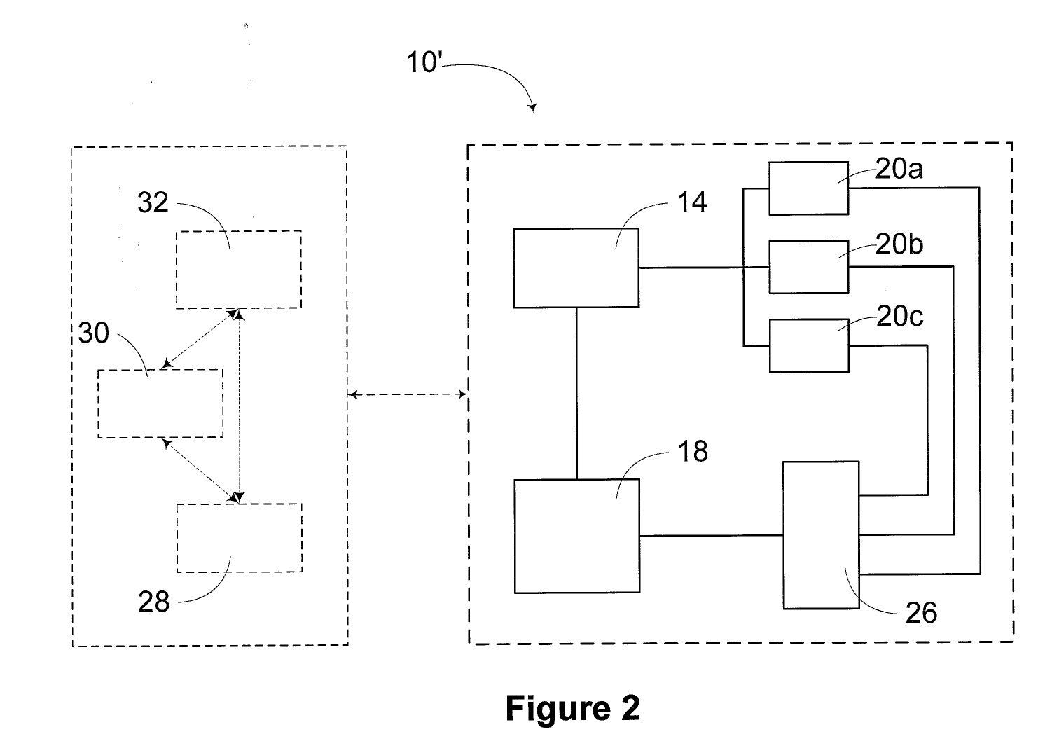

Turning now to FIG. 2, an exemplary embodiment of a radiofrequency ablation device 10′ is illustrated. In the illustrated radiofrequency ablation device 10′, bold lines are used to denote a primary circuit, and dashed lines are used to denote external devices that interact with the primary circuit. That being said, it must be understood that the terms “primary circuit” and “external devices” are used herein for clarity and ease of description, only. It is entirely possible to embody many or all of the “external devices” in the “primary circuit” devices without departing from the scope of the appended claims. However, it is easier to understand the interaction between the various functions/devices of the radiofrequency ablation device 10′ using the chosen depiction methods.

A radiofrequency generator 18 is electrically coupled to an electrode 14. The electrode 14 is electrically coupled to the conductors, illustrated here as 20a, 20b, and 20c. It should be understood that although the illustrated exemplary radiofrequency ablation device shows three conductors 20a, 20b, 20c operating in parallel, there can be more or less than three conductors, and the group of conductors need not operate solely in parallel. Rather, some or all of the conductors, for example, conductor 20c, could be replaced with two or more conductors operating in series or parallel (not illustrated). Additionally, the conductors could operate in series or in conjunction with other components (not shown). As will be understood, therefore, the number of conductors can be varied and each conductor can be controlled by the radiofrequency ablation device 10′.

The conductors 20a, 20b, 20c can be coupled to a switching circuit 26. The switching circuit 26 can be, for example, a relay, a limit switch, an electronic control circuit, or any other suitable device. The switching circuit 26 is coupled to the radiofrequency generator 18. It should be understood that additional components can be included in the primary circuit. In the illustrated exemplary embodiment, however, the additional components are shown as external devices 28, 30, and 32 for ease of description. As depicted by the dashed lines and arrows, the external devices 28, 30, 32 can be configured to interact with each other in any combination. The three illustrated external devices 28, 30, 32 can pass data to and from the primary circuit components, and to and from each other as needed.

A data acquisition device 28, for example, can interact with the conductors 20a, 20b, 20c by tracking, for example, temperature readings at the conductors 20a, 20b, 20c, or the electrodes 14. The data acquisition device 28 can also track current passing through the conductors 20a, 20b, 20c, or the electrode 14. The data acquired by the data acquisition device 28 can be passed to any other external device, for example, a controller 30. The controller 30 can be, for example, a personal computing device (PC). The controller 30 can use the acquired temperature or current data to configure the switching of the conductors 20a, 20b, 20c to optimize the radiofrequency ablation system 10′. Optimization can be determined by any number of factors, for example, minimum temperature at the conductors 20a, 20b, 20c, current flow at the conductors 20a, 20b, 20c, and the like. The controller 30 can also relay the data collected by the data acquisition device 28 to a storage component, such as a system memory (not shown). For example, the data can be stored and used later for analysis instead of, or in addition to, being utilized during a radiofrequency ablation treatment session.

In addition to the ability of the controller 30 to optionally accept and process data passed to it by the data acquisition device 28, the controller 30 can also pass a control algorithm to the switching circuit 26 without using data passed to it by the data acquisition device 28.

The controller 30 can be a PC controlled by software, as explained above. The software can include specific algorithms that can be passed to the switching circuit 26 to selectively activate or deactivate the conductors 20a, 20b, 20c. Unlike prior art systems, the exemplary radiofrequency ablation device 10′ can energize the conductors 20a, 20b, 20c in any desired combination for any desired length of time, and does not have to activate all of the conductors simultaneously or one conductor at a time in a predetermined or random sequence. Instead, the switching circuit 26 enables the radiofrequency ablation device 10′ to operate with any number of activated conductors 20a, 20b, 20c at any given time.

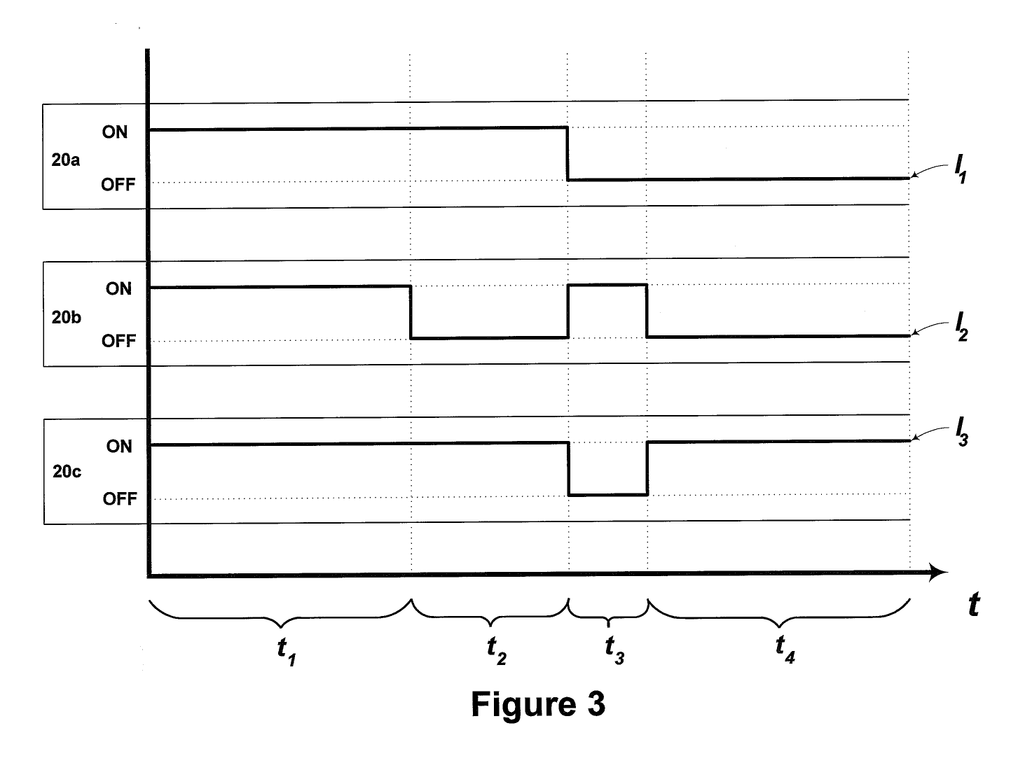

Referring briefly to FIG. 3, exemplary activation schedules for conductors 20a, 20b, 20c of an exemplary radiofrequency ablation device 10′ are illustrated. In FIG. 3, the activation schedules for respective conductors 20a, 20b, 20c are depicted by lines I₁, I₂, and I₃, respectively. For example, for a first time period (t₁), a first combination of conductors (c₁), for example, 20a, 20b, 20c can be activated. Then, for a second time period (t₂), a second combination of conductors (c₂), for example, 20a and 20c can be activated, and conductor 20b can be deactivated. Then, for a third time period (t₃), a third combination of conductors (c₃), for example, 20b can be activated, and conductors 20a and 20c can be deactivated. Then, for a fourth period of time (t₄), a fourth combination of conductors (c₄), for example, 20b and 20c can be activated, and 20a can be deactivated. The combinations c₁, c₂, c₃, and c₄ can be repeated any number of times, if desired, for time periods t₁, t₂, t₃, and t₄. It should be noted that the time periods t₁, t₂, t₃, and t₄ need not be of equal duration. Alternatively, the order of the combinations c₁, c₂, c₃, c₄, and/or the order of the time periods t₁, t₂, t₃, t₄, can be changed for a second, third, or nth permutation, where n is a positive integer. A desired cycle of combinations and/or time periods can be repeated and/or alternated for the duration of a radiofrequency ablation treatment.

It should be noted that the above combinations c₁, c₂, c₃, c₄, and time periods t₁, t₂, t₃, t₄, are nonlimiting examples provided for the purpose of teaching the principles of the invention by illustrating one of many possible methods for use with the exemplary radiofrequency ablation device 10′. For example, the radiofrequency ablation device 10′ can include any suitable number of conductors, and can activate those conductors in any desired combination, can operate for any desired number of time periods, and more can cycle through those combinations and/or time periods in a predetermined order or in random fashion. Instead, the exemplary schedule of operation should be understood as demonstrating the ability to activate any combination of conductors sequentially or simultaneously for any desired time period.

The combinations c₁, c₂, c₃, also can be determined by measurement of temperature, current, or impedance of some or all of the conductors 20a, 20b and 20c. In an exemplary embodiment using temperature, current, or impedance measurement to determine the combinations c₁, c₂, c₃, the combinations can be determined as follows: c₁ includes all conductors, c₂ includes the two conductors where lowest and second lowest temperature (or alternatively current or impedance) are recorded, and c₃ includes only the conductor where lowest temperature (or alternatively current or impedance) is recorded.

The ability to activate various combinations of conductors 20a, 20b, 20c at various times has demonstrated through clinical research and clinical trials to decrease the overall maximum heating at a given conductor 20a, 20b, 20c, and has been shown to decrease the temperature variation at respective conductors 20a, 20b, 20c. By lowering the overall maximum temperature, the incidence of skin burns can be reduced, the maximum applied power can be increased, and treatment times can be reduced, if desired. Alternatively, longer treatment times at the same applied power can be used since the incidence of skin burns can be reduced. This can result in more favorable results from treatments. Another way to further reduce heating is to switch the conductors at zero crossings of the RF signal. Switching the conductors at zero crossings of the RF signals prevents artifacts due to switching, thereby avoiding some added stimulation of excitable tissue.

Returning now to FIG. 2, a monitoring device 32 can also be included in the radiofrequency ablation device 10′. The monitoring device 32 can include, for example, a thermal camera for measuring surface temperature of the conductors 20a, 20b, 20c. The monitoring device 32 can also include monitoring systems that interface with the patient 12 receiving treatment (not illustrated in FIG. 2). For example, the monitoring device 32 can include heart rate or oxygen sensors, sphygmomanometers, respiration monitors, and the like, for monitoring the patient's condition during treatment.

The monitoring device 32 can also include a thermal sensor for monitoring conductor temperature, electrode temperature, or skin temperature during a radiofrequency ablation treatment session. The monitoring device 32 can optionally include the ability to activate cooling or heating elements, alarms, shut-off commands, combinations thereof, or the like.

Regardless of the type of external devices used, all of the external devices 28, 30, and 32 can interact with the radiofrequency ablation device 10′ if desired. The interaction with the radiofrequency ablation device 10′ can be for any purpose, including, but not limited to, optimizing radiofrequency ablation device performance during treatment, providing safety features, collecting data, or any other desired purpose.

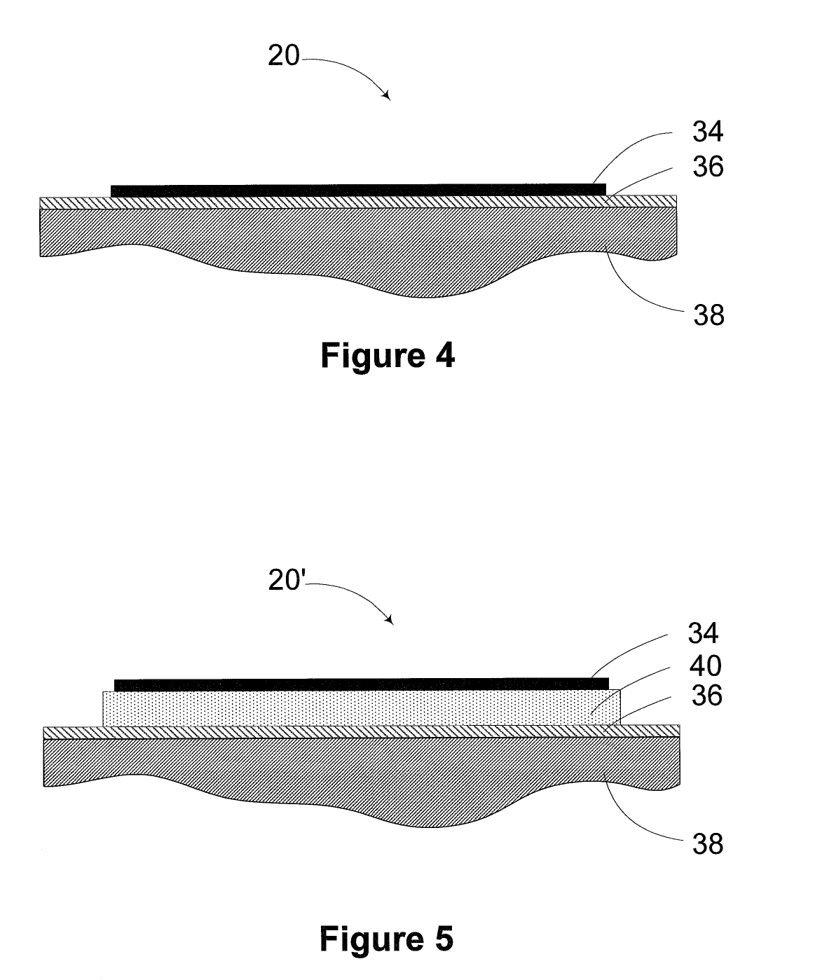

The radiofrequency ablation device 10′ of the present invention can be used with monolithic as well as composite conductors. As used herein, the term monolithic refers to a substantially unitary conductor that is formed in a single layer that as an aggregate has a given electrical conductivity. The single layer may include disparate materials that may be mixed or stratified, but functions as a single conductive element. Referring now to FIG. 4, a monolithic conductor 20 is illustrated in partial cross-section. As shown, a monolithic conductor 20 can be made from electrically conductive material. Typically, but not necessarily, the conductive material is a single layer 34 of metal foil. Though not illustrated, one surface of the layer 34 can further receive a coating of an adhesive polymer to facilitate attachment to the patient's skin 36 at a grounding site. Under the grounding site is a subcutaneous layer 38, typically a layer of fat. As is known, the illustrated view is a small cross section of a patient's body and other layers of tissue exist below the surface of the skin 36 and contiguous to both side edges of the depicted cross-section.

As an example of a composite conductor, an exemplary layered pad 20′ for use with a radiofrequency ablation device 10′ according to the present invention is illustrated in FIG. 5.

As was briefly explained above, the conductivity of tissue and other materials can vary greatly. For example, fat tissue can have lower electrical conductivity than skin tissue. Because of this variance in electrical conductivity, different tissues can effect more or less conversion of power to heat due to resistive heating. Since skin can have comparatively higher electrical conductivity than fat, it is possible that tissue conductivity is an additional cause of higher current density at the leading edge of a conductor. Furthermore, the thickness of subcutaneous fat layers can vary, not only among different patients, but also at different parts of the body of a single patient. This can make conductor heating less predicable from patient to patient, or from one area of a patient's body to another area of the patient's body.

Therefore, the layered conductor 20′ can further reduce heating experienced at its leading edge. As illustrated, a layered conductor 20′ can include at least two layers of electrically conductive material. A first layer 34 of the layered conductor 20′ can be a layer of metal foil, for example copper foil. The first layer 34 of metal foil can be substantially similar to the metal foil used as the ground pad conductor 20. Additionally, the layered conductor 20′ can further include a second layer 40 of electrically conductive material. As illustrated, the first and second layers 34, 40 do not need to have identical dimensions. In fact, by allowing the second layer 40 to have larger dimensions than the first layer 34, it can be possible to further reduce heating at the leading edge of the conductor 20′.

If desired, the second layer 40 of electrically conductive material can be of higher electrical conductivity than the adjacent tissue layer, typically the patient's skin 36, but lower electrical conductivity than the first layer 34. For example, the second layer 40 of material can be a layer of gel made of agar-water. As an example, an agar-water gel with electrical conductivity of approximately 32 S/m has about 80 times the conductivity of muscle tissue, which has an approximate conductivity of about 0.4 S/m. Copper metal foil can have electrical conductivity of from about 58,000,000 to 59,600,000 S/m. By including a layer 40 of electrically conductive material between the patient and the highly conductive metal foil layer 34, some of the resistive heating can be moved from the patient's skin 36 to the second layer 40 of the layered conductor 20′.

The second layer 40 of material can be of any desired thickness, including from 1-5 mm, and any desired conductivity, including 2 to 20 times the conductivity of the adjacent tissue, typically the patient's skin 36. For example, the second layer 40 can have twice the electrical conductivity of the patient's skin 36 and can be 1 mm thick. As another example, the second layer 40 can have twenty times the conductivity of the patient's skin 36 and can be 5 mm thick. Layered conductors 20′ can be used depending upon factors such as the patient's physical attributes.

By including a second layer 40 having different specifications, including size, shape, thickness, conductivity, and material type, in the layered conductor 20′, the point of higher current density, i.e., the leading edge of the metal foil layer 34, can be moved away from the patient's skin 36. Therefore, the use of a layered conductor 20′ can further reduce the incidence and/or the severity of skin burns, especially when used with a radiofrequency ablation according to an exemplary embodiment of the present invention.

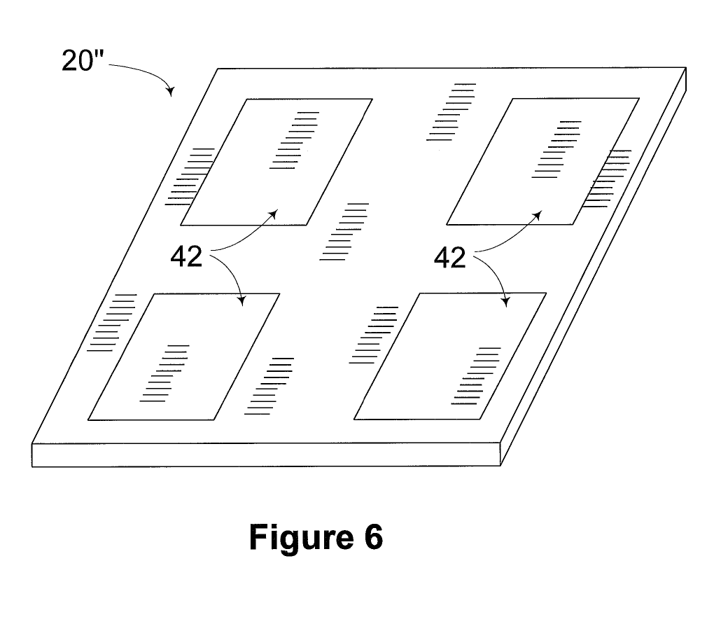

Turning now to FIG. 6, a perspective view of an exemplary a multiple-zone conductor pad 20″ according to an exemplary embodiment of the present invention is illustrated.

The multiple zone conductor pad 20″ can include any number of conductive zones 42. Although not illustrated, the multiple zone conductor pad 20″ can also include other components, such as, for example, lead wires, monitoring equipment, sensors, and the like. The conductive zones 42 can be activated individually, sequentially, and/or in combination for any desired time. In other words, the conductive zones 42 can be used in a manner substantially similar to the manner used to activate individual conductors 20, 20′, 20a, 20b, 20c.

Although the multiple zone conductor pad 20″ is shown with four conductive zones 42, it should be understood that this is not necessarily the case. Instead, any number of conductive zones 42 can be included in the multiple zone conductor pad 20″. Additionally, although the conductive zones 42 are illustrated as having equal dimensions, it should be understood that the conductive zones 42 need not have the same dimensions or shape. Furthermore, although there are no additional hardware components illustrated, it should be understood that additional hardware components, e.g., temperature sensors, relays, lead wires, monitoring equipment, or the like, can be included in the multiple zone conductor pad 20″, or at any or all of the conductive zones 42.

### EXAMPLES

The following examples are put forth so as to provide those of ordinary skill in the art with a more complete understanding of how the disclosed system can be operated. These examples are intended to be purely exemplary and are not intended to limit the scope of the appended claims. Efforts have been made to ensure accuracy with respect to the various measurements, e.g., distances, temperatures, percentages, margins of error, and the like, but some errors and deviations may not be represented in the measurements provided.

### Exemplary Radiofrequency Ablation Device

### Setup

A large plastic bath was filled with 0.25% saline to a depth of 8 cm. As is known, 0.25% saline has substantially the same electrical conductivity as muscle tissue at radiofrequency frequencies. At one end of the bath, a stainless steel electrode was placed to deliver radiofrequency energy. An agar-water gel (5% agar, 0.25% saline) block measuring 35 cm long×20 cm wide×2 cm thick was placed on top of smaller blocks of agar-water gel blocks such that the large block was only partially submerged in the saline bath. As set up, substantially all of the large block protruded from the saline bath. Three thin copper sheets with dimensions of 10 cm long×5 cm wide were placed on the protruding surface of the large agar-water gel block to serve as the ground pads. The leading edge of the first pad was located approximately 30 cm from the electrode. The leading edge of the first pad (hereinafter “proximal pad”) was the closest conductive material relative to the electrode. The leading edge of the second pad (hereinafter “middle pad”) was located 4 cm from the end of the proximal pad, and the leading edge of the third pad (hereinafter “distal pad”) was located 4 cm from the end of the middle pad. As such, the leading edge of the distal pad was approximately 58 cm from the electrode.

A radiofrequency generator, for this example, an ADVANCED ENERGY® PDX-500, was used at a frequency of 350 kHz to supply radiofrequency energy to the system at a constant power of 100W. Software was developed to control the power delivery to the system and to run the switching program.

A relay switching circuit interfaced with the software via a data acquisition device, for this example, an AGILENT® 34970A. From preliminary experiments, it was determined that the appropriate switching times (tₐ, t_{b}, and t_{c}) were 700, 800 and 550 milliseconds, respectively. Table 1 below schematically represents approximate activation times in seconds (s) for the ground pads switched in this example.

### Trials

A total of twelve 12-minute trials were performed. For six trials, the pads were activated contemporaneously, and for six trials, the switching circuit was used to sequentially activate different combinations of the pads (hereinafter “switched activation”).

### Results

The leading edge temperatures of each of three pads in simultaneous activation were significantly different (p<0.0001), while the leading edge temperature of the pads in the switched activation trials were more uniform (p=0.07). Overall, the maximum temperature of the proximal pad during simultaneous activation was significantly higher than during switched activation; 10.7±1.0° C. versus 4.8±0.2° C.). At the middle and distal pads, the temperature was higher using simultaneous activation than when using switched activation.

### Exemplary Layered Pad

A clinical trial of a prototype layered conductor 20′ included a 5 mm thick agar-water gel layer with a conductivity of approximately 32 S/m for the second layer 36. Copper foil was used for the first layer 34. A 1.5 A radiofrequency current at 375 kHz was applied for six minutes. Then, a monolithic metal foil conductor 20 was used during a substantially identical trial. The layered pad 20′ resulted in approximately 68% lower maximum temperature rise compared to the monolithic conductor 20.

The law does not require and it is economically prohibitive to illustrate and teach every possible embodiment of the present claims. Hence, the above-described embodiments are merely exemplary illustrations of implementations set forth for a clear understanding of the principles of the invention. Variations, modifications, and combinations may be made to the above-described embodiments without departing from the scope of the claims. All such variations, modifications, and combinations are included herein by the scope of this disclosure and the following claims.

What is claimed is:

1. A radiofrequency ablation device, comprising:
at least one electrode for being located at an ablation site of a patient receiving radiofrequency ablation treatment;
a plurality of conductors electrically coupled to the at least one electrode, and located at a grounding site on the patient;
a power supply electrically coupled to the electrode; and
a switching circuit electrically coupled to the conductors; wherein
at any time during a radiofrequency ablation treatment session, the switching circuit selectively activates or deactivates any number of the of conductors,
for at least a portion of the radiofrequency ablation treatment session, at least two conductors are simultaneously activated, and
for at least a second portion of the radiofrequency ablation treatment session, at least one conductor is deactivated.

2. The radiofrequency ablation device of claim 1, wherein there are at least three conductors.

3. The radiofrequency ablation device of claim 1, wherein each of the plurality of conductors further comprises a plurality of layers of electrically conductive material.

4. The radiofrequency ablation device of claim 3, wherein the plurality of layers of electrically conductive material comprise a first layer and a second layer.

5. The radiofrequency ablation device of claim 4, wherein the first layer of electrically conductive material is more electrically conductive than the second layer of electrically conductive material.

6. The radiofrequency ablation device of claim 5, wherein the second layer of electrically conductive material that is more electrically conductive is disposed proximal to a skin layer of the patient receiving radiofrequency ablation treatment.

7. The radiofrequency ablation device of claim 1, further comprising a control system operatively coupled to the switching circuit, wherein the control system is for executing computer readable instructions that control operation of the switching circuit.

8. The radiofrequency ablation device of claim 1, wherein the radiofrequency ablation device further comprises a monitoring system.

9. The radiofrequency ablation device of claim 8, further comprising a control system operatively coupled to the switching circuit, wherein the monitoring system obtains data relating to the radiofrequency ablation device, and the control system controls operation of the switching circuit based, at least partially, on the data obtained by the monitoring system.

10. The radiofrequency ablation device of claim 8, wherein the monitoring system is a temperature sensor.

11. The radiofrequency ablation device of claim 8, wherein the monitoring system is a current sensor.

12. The radiofrequency ablation device of claim 8, wherein the monitoring system is an impedance sensor.

13. The radiofrequency ablation device of claim 1, wherein the switching circuit is a relay.

14. The radiofrequency ablation device of claim 1, wherein the switching circuit activates or deactivates the conductors at zero-crossings of the radiofrequency signal.

15. A method for reducing the incidence of skin burns during radiofrequency ablation treatment, comprising:
locating at least one electrode at an ablation site of a patient receiving radiofrequency ablation treatment;
electrically coupling a plurality of conductors to the at least one electrode; and
locating each of the conductors at a grounding site on the patient;
electrically coupling a power supply to the at least one electrode; and
electrically coupling a switching circuit to the conductors; wherein
at any time during a radiofrequency ablation treatment session, the switching circuit can selectively activate or deactivate any number of the conductors,
for at least a portion of the radiofrequency ablation treatment session, at least two conductors is simultaneously activated, and
for at least a second portion of the radiofrequency ablation treatment session, at least one conductor is deactivated.

16. The method of claim 15, wherein there are at least three conductors.

17. The method of claim 16, wherein each of the conductors further comprises a plurality of layers of electrically conductive material, a first layer and a second layer.

18. A computer-readable medium comprising computer-executable instructions that, when executed, perform the method of claim 15.
